(19) United States
(12) Patent Application Publication
(10) Pub. No.: US 2018/0014770 A1
(43) Pub. Date: Jan. 18, 2018

(54) Measurement Device, Measurement Tube and the Use Thereof for Monitoring of Urine Flow

(71) Applicant: Drugster ApS, Aarhus (DK)

(72) Inventor: Ulrik Merrild NIELSEN, Brabrand (DK)

(73) Assignee: Drugster ApS, Aarhus (DK)

(21) Appl. No.: 15/536,074

(22) PCT Filed: Dec. 14, 2015
(86) PCT No.: PCT/DK2015/050392

§ 371 Date: Jun. 14, 2017

(30) Foreign Application Priority Data
Dec. 15, 2014 (DK) PA201470784

(51) Int. Cl. A61B 5/20; A61B 5/00; A61L 29/04; A61B 5/145

(52) U.S. Cl. CPC A61B 5/208; A61L 29/041; A61B 5/0075; A61B 5/6852; A61B 5/14507

(57) ABSTRACT

A measurement device (11) is disclosed for measurement of a flow of urine (7) in a flexible tube (4), the device (11) comprising jaws (12, 13) arranged for receiving a section of the tube (4), the device (11) further comprises an arrangement (14, 15, 6) for directing light through the tube (4) in said first direction so as to perform said measurement. A measurement tube (20) is also disclosed having a measurement area (22) within substantially parallel inner walls (23) with a distance in the range of to 4 millimetres as well as the use of the measurement tube (20) with the device (11).

The present invention relates to a measurement device for monitoring flow of urine through a urine catheter assembly in order to provide point-of-care monitoring of patients, a measurement tube for the same use as well as the use of such measurement device with the measurement tube.

### BACKGROUND

In the treatment of patients, in particular of patients having kidney-related deceases, the medical personnel have a need for monitoring the contents and concentration in the urine of a number of elements, such as carbamide, creatinine, sodium ions and/or potassium in urine. Similarly, examination may be of content of albumine, glucose, nitrogen and/or oxygen to investigate other functions of the body, or whether the urine contains myoglobin, blood or glucose, which may be indicative of various disease states. Also traces of medical components in the urine may be of importance to evaluate the state of a patient's condition. Point-of-care monitoring and testing of patients, that be hospitalized patients or patients at home is of increasing importance as it may reduce the requirement of laboratory analysis of test samples of e.g. urine from the patients, provides in general results more rapidly that laboratory analysis and provides the test results near the patient and the care givers so that action can be taken immediately, if necessary.

In order to obtain a widespread use of point-of-care monitoring, it is desirable to provide simple-to-use devices of reasonable costs for each device as the number of devices increases drastically as compared to test facilities for laboratory use.

### DESCRIPTION OF THE INVENTION

Disclosed herein is a measurement tube for a urine catheter assembly, the measurement tube having an inner cross-sectional opening with a measurement area having substantially parallel inner walls and a height of the measurement area being the distance between said walls in the range of 1 to 4 millimetres, preferably in the range of 1.3 to 2.5 millimetres. Hereby, the measurement tube is suitable for being used for optical measurement of the contents of the urine without requiring the measurement tube to be compressed or only be compressed slightly to achieve an optical path through the measurement area that is sufficiently short for a reliable measurement to be made. A compression of a flexible tube is demanding on the mechanical properties of the tube to avoid the formation of cracks in the tube, and the present measurement tube is thus advantageous over the known tubes with an inner opening of a substantially circular cross-section and a typically inner diameter of about 6 millimetres.

The measurement area has preferably an extent parallel to the inner walls in the range of 4 to 12 millimetres, preferably in the range of 6 to 10 millimetres so as to provide a suitable cross-sectional area for optical measurements.

The tube is preferably made in a flexible material but may also be manufactured in a rigid material.

The preferred material of the measurement tube is polytetrafluoroethylene (PTFE).

In a preferred embodiment, the inner cross-sectional opening of the measurement tube further comprises a flow area, which preferably is a substantially circular area, of an extend preferably in the range of 10 to 40 square millimetres. The function of the flow area is to ensure that measurement tube can accommodate the full flow of urine from a patient.

The flow area may in a particular embodiment be separated from the measurement area by an air drain channel extending in the longitudinal direction of the tube and being provided with an opening or openings towards the measurement area so as to ensure that the measurement area at the beginning of the use of the measurement tube may be drained of air and filled with urine.

The present invention also relates to the use of such measurement tube in an catheter assembly connecting a catheter in a patient with e.g. a urine drainage bag where an optical measurement of the characteristics of the urine is performed through the measurement area.

The present invention also relates to a measurement device for measurement of a flow of urine in a measurement tube as described herein, the device comprising two substantially parallel jaws forming there between a groove for receiving a part of the measurement tube comprising the measurement area, the device further comprises in one of said jaws an arrangement for directing light through said tube part in a first direction substantially perpendicularly to a longitudinal direction of the tube so as to perform said measurement, the device further comprising a light detector in the other of said jaws arranged to receive light directed through said tube part by means of said arrangement.

The device may comprise mutually movable jaws arranged for receiving a section of the tube and either compressing the tube in a first direction substantially perpendicularly to a longitudinal direction of the tube, so that the height of the inner opening in the tube is reduced in the first direction, or just to capture and hold the measurement tube, e.g. a rigid embodiment of the measurement tube.

The device comprises in a preferred embodiment furthermore an optical beam expander arranged to expand light prior to being directed through the tube so as to eliminate or reduce the disturbing effect of air bubbles or solid or semisolid particles in the urine. In particular, it has been found by the present inventor that the optical beam expander preferably is arranged to expand the incoming light to a beam diameter in the range of 2.5 to 8 millimetres, preferably to a diameter in the range of 3.5 to 6 millimetres, whereby the effect of in particular air bubbles is negligible.

The present invention also relates to a system comprising such measurement device together with a measurement tube according to the present invention.

Also, the present invention relates to the use of a measurement device as disclosed herein with a urine catheter assembly comprising a measurement tube according to the present invention, wherein the tube part comprising the measurement area is inserted between the jaws of the measurement device.

The measurement tube is for this use preferably a flexible tube and the tube part inserted between the jaws of the measurement device is compressed so that the height of the inner measurement area opening in the tube part is in the range of 1 to 2.5 millimetres, preferably in the range of 1.3 to 2 millimetres.

Furthermore is disclosed a measurement device for measurement of a flow of urine in a flexible tube, such as a tube with a substantially circular outer cross-section, the device comprising mutually movable jaws arranged for receiving a section of the tube and compressing the tube in a first direction substantially perpendicularly to a longitudinal direction of the tube, so that the height of the inner opening in the tube is reduced in the first direction, the device further comprises an arrangement for directing light through the tube in said first direction so as to perform said measurement. The movable jaws are mutually movable to receive and compress the section of the flexible tube, i.e. that both jaws may be movable with respect to a support part for the jaws or one of the jaws is stationary with such support part and the other jaw is movable. The jaws may be mutually movable in a scissor-like movement pattern or they may be translated towards or from each other while remaining parallel. The arrangement for directing light is preferably connected to or included in one of the jaws.

The device comprises a light detector arranged to receive light directed through the tube by means of said arrangement, said light detector being connected to or arranged in a jaw, in particular the opposite jaw as the one to which the arrangement for directing light is connected to or included in, i.e. a forward-scatter mode, which for most optical measurement methods provides the best signal-to-noise ratio, provided that the light path through the medium to be measured is not too long. Alternatively, the measurement device is arranged in a backscatter setup where the light detector may be arranged in or connected to the same jaw as the arrangement for directing light, or the detector may be situated at a remote location to which the measurement device is connected to by an optical connection, in particular by means of an optical fibre cable.

The present invention also relates to the use of such measurement device as described herein with a urine catheter assembly comprising a flexible tube, which preferably connects a catheter and a urine drainage container, where the section of the tube inserted into the measurement device is compressed by means of said jaws so that the height of the inner opening in the tube is in the range of 1 to 3 millimetres, preferably in the range of 1.3 to 2 millimetres. Hereby, the optical pathway of the light through the urine is of a magnitude so that a reasonable signal-to-noise ratio is obtainable, also in a forward-scatter arrangement.

It has turned out that an improved result of the measurements is obtained when the section of the flexible tube inserted into the measurement device is made from polytetrafluoroethylene (PTFE) which is also known by the trade name of Teflon. The whole flexible tube of the urine catheter assembly may be made from PTFE or one a subsection where the measurement device is arranged.

According to one embodiment, the flexible tube is arranged so that the section of the tube compressed by the measurement device is continuously filled with urine.

According to another embodiment, the flexible tube is arranged so that the urine will pass the measurement device drop wise. In this case, it is possible to include an automated calculation of an estimate of the flow rate of urine based on the number of drops of urine passing the measurement device per time unit.

The measurement of the urine may include use of Raman spectroscopy, which is well-known in the art for such purpose in a number of different configurations, such as surface-enhanced Raman scattering (SERS).

Alternatively or additionally, the measurement of the urine may include use of Near-infrared (NIR) spectroscopy, in particular by means of the use of three to eight substantially monochromatic light sources, in particular Laser Emitting Diodes (LED).

Alternatively or additionally, the measurement of the urine may include use of Mid-infrared (MIR) spectroscopy, in particular by means of the use of three to eight substantially monochromatic light sources, in particular Laser Emitting Diodes (LED).

The combinations of measurements using Raman spectroscopy and NIR spectroscopy and/or MIR spectroscopy are preferred, as the combination may improve the reliability of the resulting outcome of the measurements.

In a further aspect of the present invention, it relates to a measurement device for measurement of a flow of urine in a flexible tube, the device comprising a measurement unit having two parallel transparent panels defining a cavity and connectors for connecting the measurement unit with the tube, so that the flow of urine through the tube will pass through the measurement unit, wherein the inner perpendicular distance between the panels is in the range of 1 to 2.5 millimetres, preferably in the range of 1.3 to 2 millimetres, the device further comprises an arrangement for directing light through the measurement unit in a direction substantially perpendicular to the panels so as to perform said measurement.

This measurement device may further comprise a light detector arranged to receive light directed through the measurement unit by means of said arrangement

It is also preferred that this measurement device further comprise an optical beam expander arranged to expand light prior to being directed through the measurement unit, in particular being arranged to expand the incoming light to a beam diameter in the range of 2.5 to 8 millimetres, preferably to a diameter in the range of 3.5 to 6 millimetres.

The arrangements and uses disclosed above with respect to the aspect of the present invention as described first may also apply to the measurement device according to the further aspect of the present invention, i.e. that the flexible tube is arranged so that the urine will pass the measurement device drop wise, in particular for estimation of flow rate of urine, and the use of Raman, NIR and/or MIR spectroscopy.

### BRIEF DESCRIPTION OF THE DRAWINGS

Examples of the present invention is illustrated in the enclosed drawing of which

FIG. 1 shows a urinary catheter assembly with drainage bag and two areas of measurement indicated,

FIG. 2 shows a detail of the first area of measurement of FIG. 1,

FIG. 3 shows a detail of the second area of measurement of FIG. 1,

FIG. 4 shows a cross-section of a flexible tube of a urinary catheter assembly arranged in a measurement clamp according to the present invention, the cross-section being made in the cross-sectional direction of the tube,

FIG. 5 shows sketch of a measurement tube for a urine catheter assembly according to a second embodiment of the present invention,

FIG. 6 shows a cross-section of one embodiment of the measurement tube of FIG. 5,

FIG. 7 shows a cross-section of a second embodiment of the measurement tube of FIG. 5, and

FIG. 8 shows a cross-section of a measurement tube FIG. 6 of a urinary catheter assembly arranged in a measurement device according to an embodiment of the present invention, the cross-section being made in the cross-sectional direction of the tube.

### DESCRIPTION OF PREFERRED EMBODIMENTS

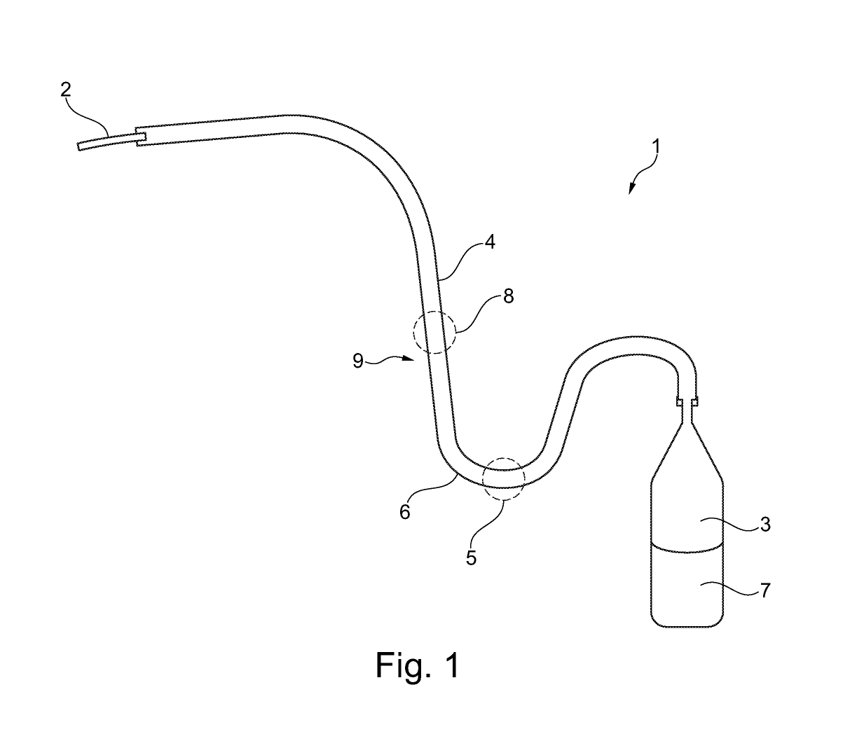

The urinary catheter assembly 1 of FIG. 1 comprises a catheter 2 and a urine drainage bag 3 connected by means of a flexible tube 4, typically of an outer diameter of about 8 millimetre. The tube 4 is in the figure arranged so that two different areas of measurement according to different aspects of the present invention are demonstrated: a first area 5 in a downwards bend 6 of the tube 4 where a portion of urine 7 will be present continuously and a second area 8 at a substantially vertical straight section 9 of the flexible tube 4 where the urine 9 will pass drop 10 by drop 10.

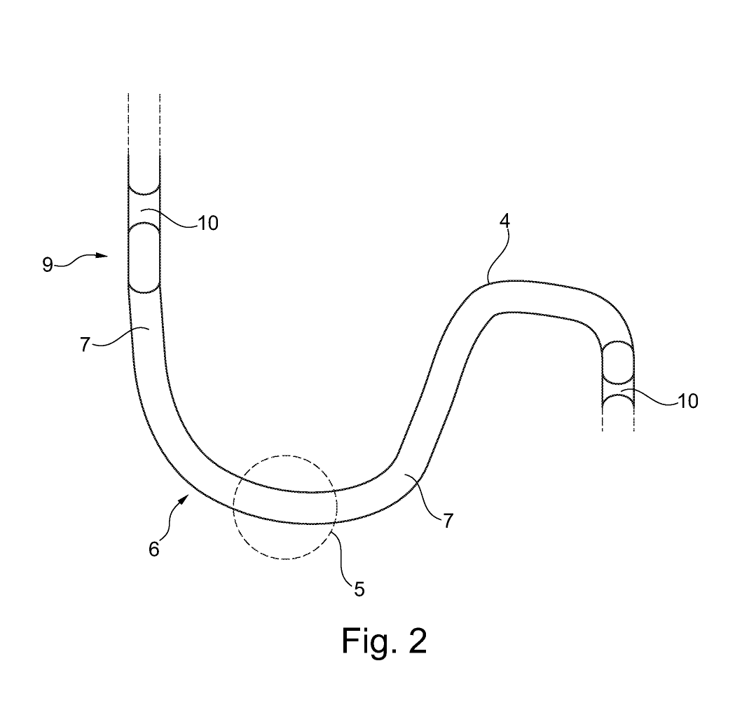

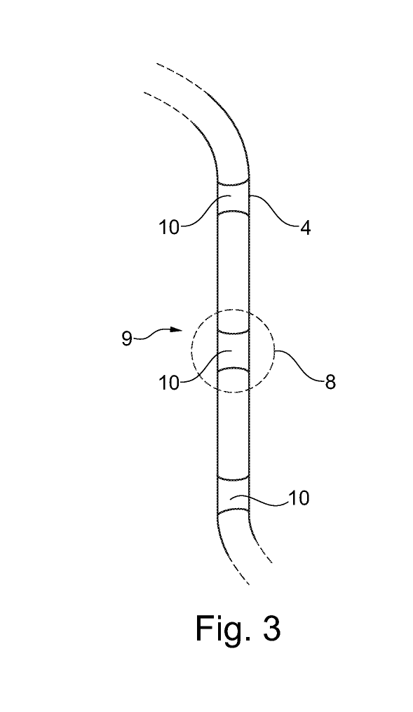

The first area 5 of measurement is shown in detail in FIG. 2, whereas the second area 8 of measurement is shown in detail in FIG. 3. In the first area 5, the urine 7 from the patient will arrive drop wise 10 into the bottom of the bend 6 of the tube 4, where an amount of urine 7 will be present continuously while the urine 6 is flowing through from the catheter 2 to the drainage bag 3. Hereby, a constant output from the measurement device 11 can be provided according to the characteristics of the urine 7 contained in the bend 6. In the second area 8, the urine 7 passes drop wise 10 from the catheter 2 to the drainage bag 3 and the measurement device 11 measures the characteristics of each drop 10 of urine and provide an output accordingly. Hereby, it is possible to count the number of drops 10 of urine 7 passing and providing an estimate of the flow rate of urine 7, based on empirical data for the type and inner diameter of the flexible tube 4.

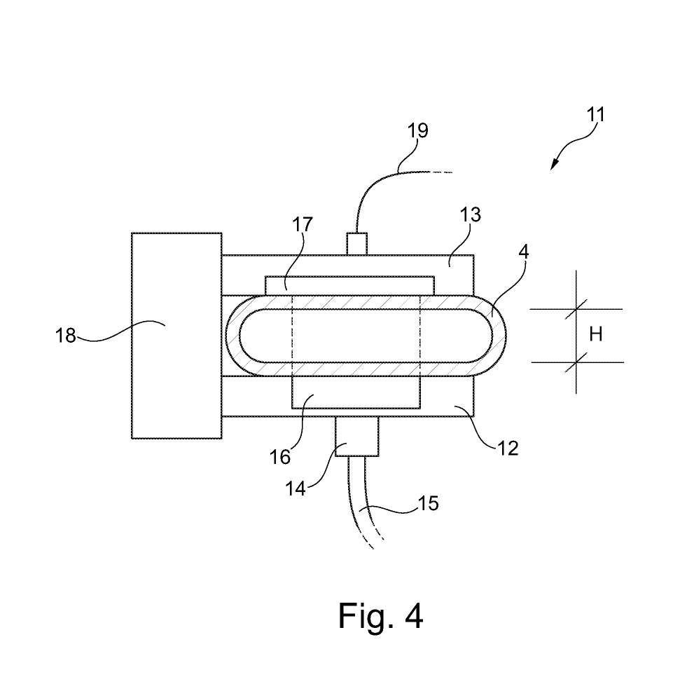

The measurement device 11 shown in FIG. 4 comprises two jaw parts 12, 13 and a support part 18 carrying the two jaw parts 12, 13 in a manner that the jaw parts 12, 13 can be moved away from each other in order to receive a piece of the flexible tube 4 between the jaws 12, 13 and towards each other to a measurement position, where the tube 4 is clamped between the jaws 12, 13 and is compressed to a flattened cross-sectional shape as shown in FIG. 4, where the height H of the inner opening of the tube 4 is about 1.5 millimetres (typically between 1 and 2 millimetres). One jaw 12 includes a connector 14 for connecting an optical fibre cable 15 to the jaw 12 and an optical beam expander 16 arranged so that the light transmitted from the optical fibre cable 15 is expanded to have a diameter of 3 to 6 millimetres when passing the measurement area 5, 8 of the tube 4. On the opposite jaw 13 is a light detector 17 arranged for detection of light from the optical beam expander 16 transmitted through the measurement area 5, 8 of the tube 4.

By compressing the piece of flexible tube 4 between the jaws 12, 13 a sufficiently short optical pathway through the possible urine 7 inside the tube 4 for the light from the optical beam expander 16 and to the light detector 17 that an acceptable signal to noise ratio is obtained. The signal from the light detector 17 is transmitted to a signal processor (not shown) with a signal cable 19.

The optical fibre cable 15 is connected to a light source box (not shown) that contains the laser source for Raman spectroscopy of the urine 6 in the measurement area 5, 7 in a forward-scatter configuration as well as the monochromatic light emitters, in particular Light Emitting Diodes (LED) employed for spectroscopy in particular in the Near Infrared (NIR) area, defined as infrared light of wavelengths between 800 nm and 2,500 nm or the Mid Infrared (MIR) area, defined as infrared light of wavelengths between 2,500 nm and 20,000 nm. In a particular embodiment of the present invention, the Raman spectroscopy is arranged in a back-scattering arrangement, and a suitable detection system for the back-scatter is placed in the light source box. By combining the use of Raman spectroscopy and NIR or MIR spectroscopy, the error margin of the measurement device 11 can be substantially reduced.

Such an apparatus for the analysis of constituents in the urine makes possible a simple and cost-effective possibility of establishing a continuous monitoring of persons with a catheter connected to their bladders. By the analysis, the function of the kidneys may be examined, for example by measuring the content of carbamide, creatinine, sodium ions and/or potassium in urine. Similarly, examination may be of content of albumine, glucose, nitrogen and/or oxygen to investigate other functions of the body, or whether the urine contains myoglobin, blood or glucose, which may be indicative of various disease states. By this simple and inexpensive solution it is achieved that analysis of urine can be done to a greater extent where the patient is located, for example in his home, and a doctor, or other health professional staffing, can at once obtain a measurement result and initiate treatment or other measures as a result.

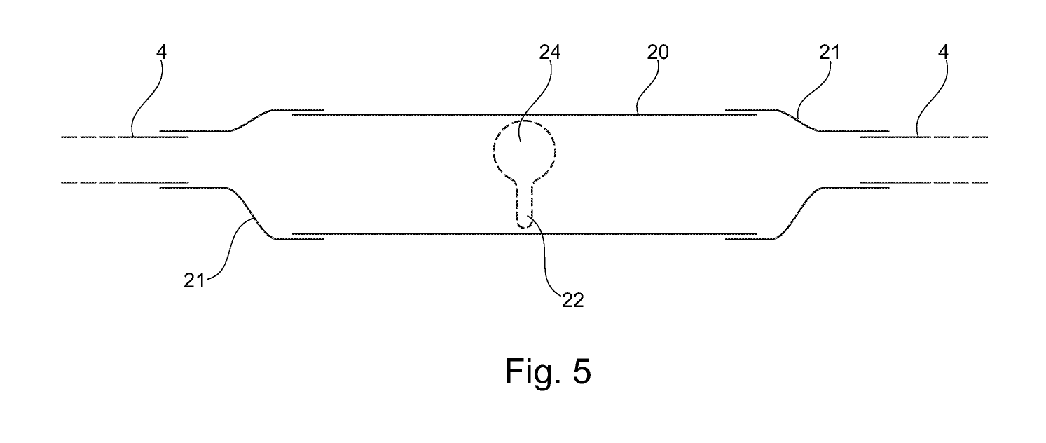

A measurement tube 20 according to the present invention is shown in FIGS. 5, 6 and 7, where FIG. 5 is a sketch of a side view of the measurement tube 20 connected to a flexible tube 4 at both ends by means of adapter tube pieces 21 that provides the transition between the substantially circular outer cross-sectional shape of the flexible tubes 4 and the outer keyhole-shape of the measurement tube 20. The cross-sectional shape of the inner opening 22, 24 is indicated on the measurement tube.

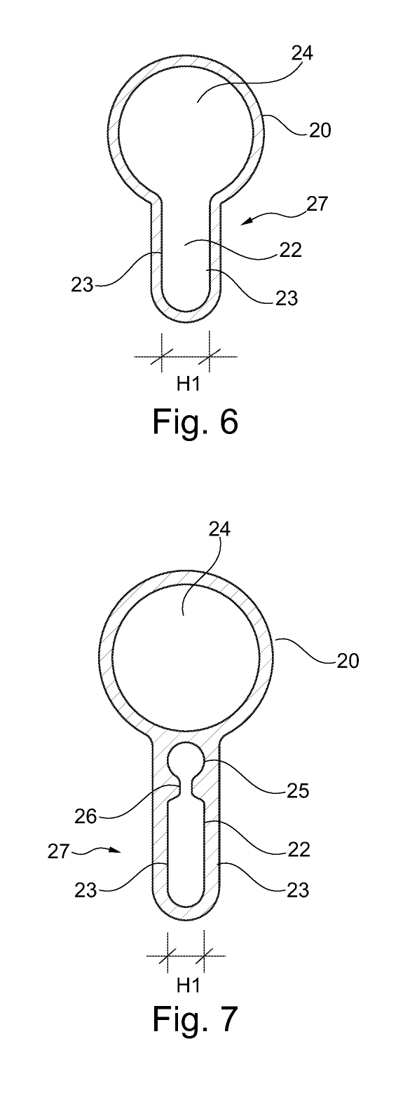

The cross-sectional shape of a first embodiment of the measurement tube 20 is shown in FIG. 6, having an inner measurement area opening 22 of a width H1 of about 2 millimetres between substantially parallel inner walls 23 and a flow area 24 of substantially circular cross-sectional shape. The tube part comprising the measurement area 22 is generally referred to with reference number 27. The measurement tube 20 is made in a flexible and transparent material, preferably in polytetrafluoroethylene. The flow area 24 is provided to ensure that the full flow of urine 7 from the patient will be able to pass through the measurement tube, whereas the measurement area opening 22 is provided for allowing optical measurement of the characteristics of the urine as discussed previously through the tube part 27 comprising the measurement area 22.

The cross-sectional shape of a second embodiment of the measurement tube 20 is shown in FIG. 7, which deviates from the first embodiment shown in FIG. 6 in that an air drain channel 25 is provided, which is connected to the measurement area opening 22 by means of one or more openings 26 provided there between. The opening or openings 26 may either be a continuous slit extending the full length of the measurement tube 20 or it may comprise a plurality of separate openings 26 with e.g. 10 to 15 millimetres separation in the longitudinal direction of the measurement tube 20. The air drain channel 25 is typically closed at the end of the measurement tube 20 connected to the catheter side of a catheter assembly and is open at the opposite end. The measurement area opening 22 as well as the flow area 24 are open at both ends of the measurement tube 20, thus allowing urine 7 to flow through both areas 22, 24. The air drain channel 25 ensures that air trapped inside the measurement area 22, in particular when the assembly is taken into use, will be drained out and allow a flow of urine 7 in the measurement area 22.

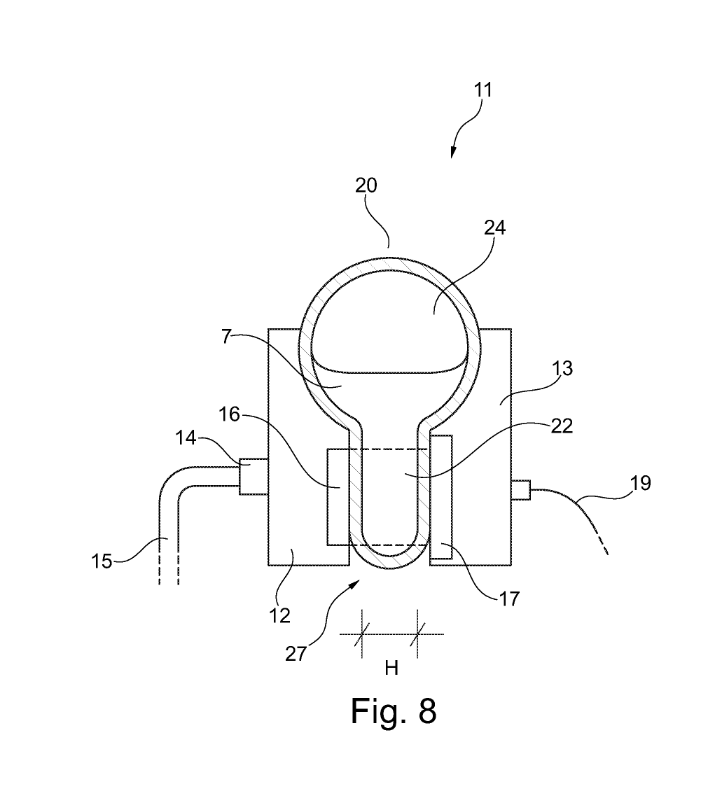

A modified version of the measurement device 11 of FIG. 4 designed to receive the measurement tube 20 of FIGS. 5 to 7 is shown in FIG. 8. The two jaws 12, 13 are of a cross-sectional shape and mutual distance to form a groove there between for receiving at least a part of the tube part 27 comprising the measurement area 22 so that light from the optical fibre cable 15 can be transmitted through the urine 7 in the measurement area 22 to the light detector 17. The jaws 12, 13 may either be of a fixed mutual distance, so that the tube part 27 may be manually pushed into the groove, or the distance between the jaws 12, 13 may be adjustable so that the tube part 27 is first inserted between the jaws 12, 13 and then compressed by moving the jaws 12, 13 towards each other. The device 11 is oriented so that the tube part 27 containing the measurement area 22 is downwards in order to ensure that it is filled with urine even in situations where the whole inner opening 22, 24 is not, as indicated in FIG. 8, where only the lower half of the circular flow area 24 is filled with urine.

### REFERENCES

1 Urine catheter assembly
2 Catheter
3 Urine drainage bag
4 Flexible tube
5 First measurement area
6 Downwards bend of the tube
7 Urine
8 Second measurement area
9 Straight section of the tube
10 Urine drop
11 Measurement device
12 First jaw of device
13 Second jaw of device
14 Connector
15 Optical fibre cable
16 Optical beam expander
17 Light detector
18 Support part
19 Signal cable
20 Flexible measurement tube
21 Adapter tube piece
22 Measurement area opening
23 Inner walls of measurement area
24 Circular flow area opening of measurement tube
25 Air drain channel
26 Opening between air drain channel and measurement area opening
27 Tube part comprising the measurement area
H Inner height of cross-sectional opening in tube compressed in measurement device
H1 Inner height of cross-sectional measurement area opening in measurement tube

1. Measurement tube (20) for a urine catheter assembly (1) having an inner cross-sectional opening with a measurement area (22) having substantially parallel inner walls (23) and a height (H1) of the measurement area (22) being the distance between said walls (23) in the range of 1 to 4 millimetres, preferably in the range of 1.3 to 2.5 millimetres.

2. Measurement tube (20) according to claim 1, wherein said measurement area (22) has an extent parallel to the inner walls (23) in the range of 4 to 12 millimetres, preferably in the range of 6 to 10 millimetres.

3. Measurement tube (20) according to claim 1 or 2, wherein the tube is made in a flexible material.

4. Measurement tube (20) according to any of claims 1 to 3, which is made from polytetrafluoroethylene (PTFE).

5. Measurement tube (20) according to any of claims 1-3, wherein the inner cross-sectional opening further comprises a flow area (24), which preferably is a substantially circular area, of an extend preferably in the range of 10 to 40 square millimetres.

6. Measurement tube (20) according to claim 5, wherein the flow area (24) is separated from the measurement area (22) by an air drain channel (25) extending in the longitudinal direction of the tube and being provided with an opening or openings (26) towards the measurement area (22).

7. Measurement device (11) for measurement of a flow of urine (7) in a measurement tube (20) according to any of claims 1 to 6, the device (11) comprising two substantially parallel jaws (12, 13) forming there between a groove for receiving a part (27) of the measurement tube (20) comprising the measurement area (22), the device (11) further comprises in one of said jaws (12) an arrangement (14, 15, 16) for directing light through said tube part (27) in a first direction substantially perpendicularly to a longitudinal direction of the tube (20) so as to perform said measurement, the device further comprising a light detector (17) in the other of said jaws (13), arranged to receive light directed through said tube part (27) by means of said arrangement (14, 15, 16).

8. Measurement device (11) according to claim 7, further comprising an optical beam expander (16) arranged to expand said light prior to being directed through said tube part (27).

9. Measurement device according to claim 8, wherein the optical beam expander (16) is arranged to expand the incoming light to a beam diameter in the range of 2.5 to 8 millimetres, preferably to a diameter in the range of 3.5 to 6 millimetres.

10. Use of a measurement device (11) according to any of claims 7 to 9 with a urine catheter assembly (1) comprising a measurement tube (20) according to any of claims 1 to 6, wherein the tube part (27) comprising the measurement area (22) is inserted between the jaws (12, 13) of the measurement device (11)

11. Use according to claim 10, where the measurement tube (20) is a flexible tube and the tube part (27) inserted between the jaws (12, 13) of the measurement device (11) is compressed so that the height (H) of the inner measurement area (22) opening in the tube part (27) is in the range of 1 to 2.5 millimetres, preferably in the range of 1.3 to 2 millimetres.

12. Use according to claim 10 or 11, wherein the measurement tube (20) is arranged so that the tube part (27) inserted between the jaws (12, 13) of the measurement device (11) is continuously filled with urine (7).

13. Measurement device (11) for measurement of a flow of urine (7) in a flexible tube (4), the device (11) comprising mutually movable jaws (12, 13) arranged for receiving a section of the tube (4) and compressing the tube (4) in a first direction substantially perpendicularly to a longitudinal direction of the tube (4), so that the height (H) of the inner opening in the tube (4) is reduced in the first direction, the device (11) further comprises in one of said jaws (12) an arrangement (14, 15, 16) for directing light through the tube (4) in said first direction so as to perform said measurement, the device further comprising a light detector (17) arranged to receive light directed through the tube (4) by means of said arrangement (14, 15, 16), wherein the light detector (17) is arranged in one of said jaws (12, 13), preferably in the other of said jaws (13).

14. Measurement device (11) according to claim 13, further comprising an optical beam expander (16) arranged to expand light prior to being directed through the tube (4).

15. Measurement device according to claim 14, wherein the optical beam expander (16) is arranged to expand the incoming light to a beam diameter in the range of 2.5 to 8 millimetres, preferably to a diameter in the range of 3.5 to 6 millimetres.

16. Use of a measurement device (11) according to any of claims 13 to 15 with a urine catheter assembly (1) comprising a flexible tube (4), where the section of the tube (4) inserted into the measurement device (11) is compressed so that the height (H) of the inner opening in the tube (4) is in the range of 1 to 2.5 millimetres, preferably in the range of 1.3 to 2 millimetres.

17. Use according to claim 16, wherein the section of the flexible tube (4) inserted into the measurement device (11) is made from polytetrafluoroethylene (PTFE).

18. Use according to claim 16 or 17, wherein the flexible tube (4) is arranged so that the section of the tube (4) compressed by the measurement device (11) is continuously filled with urine (7).

19. Use according to claim 16 or 17, wherein the flexible tube (4) is arranged so that the urine (7) will pass the measurement device (11) drop wise (10).

20. Use according to claim 19, further comprising the calculation of an estimate of the flow rate of urine (7) based on the number of drops (10) of urine (7) passing per time unit.

21. Use according to any of claims 10 to 12 or any of claims 16 to 20, wherein the measurement includes use of Raman spectroscopy.

22. Use according to any of claims 10 to 12 or to any of claims 16 to 21, wherein the measurement includes use of Near-infrared (NIR) spectroscopy.

23. Use according to claim 22, wherein said Near-infrared (NIR) spectroscopy includes the use of three to eight substantially monochromatic light sources, in particular Laser Emitting Diodes (LED).

24. Use according to any of claims 10 to 12 or to any of claims 16 to 23, wherein the measurement includes use of Mid-infrared (MIR) spectroscopy.

25. Use according to claim 24, wherein said Mid-infrared (MIR) spectroscopy includes the use of three to eight substantially monochromatic light sources, in particular Laser Emitting Diodes (LED).

26. Measurement device (11) for measurement of a flow of urine (7) in a flexible tube (4), the device (11) comprising a measurement unit having two parallel transparent panels defining a cavity and connectors for connecting the measurement unit with the tube (4), so that the flow of urine (7) through the tube (4) will pass through the measurement unit, wherein the inner perpendicular distance between the panels is in the range of 1 to 2.5 millimetres, preferably in the range of 1.3 to 2 millimetres, the device (11) further comprises an arrangement (14, 15, 16) for directing light through the measurement unit in a direction perpendicular to the panels so as to perform said measurement.

27. Measurement device (11) according to claim 26, further comprising a light detector (17) arranged to receive light directed through the measurement unit by means of said arrangement (14, 15, 16).

28. Measurement device (11) according to claim 26 or 27, further comprising an optical beam expander (16) arranged to expand light prior to being directed through the measurement unit.

29. Measurement device according to claim 28, wherein the optical beam expander (16) is arranged to expand the incoming light to a beam diameter in the range of 2.5 to 8 millimetres, preferably to a diameter in the range of 3.5 to 6 millimetres.
